(19) United States
(12) Patent Application Publication
(10) Pub. No.: US 2010/0198228 A1
(43) Pub. Date: Aug. 5, 2010

(54) A Jig

(75) Inventor: Brian Thornes, Dublin (IE)

Correspondence Address: GREENLEE WINNER AND SULLIVAN P C, 4875 PEARL EAST CIRCLE, SUITE 200, BOULDER, CO, 80301, US

(73) Assignee: SOTA Orthopaedics Limited, Malahide (IE)

(21) Appl. No.: 12/670,627

(22) PCT Filed: Jul. 24, 2008
(86) PCT No.: PCT/EP08/06090

§ 371 Date: Jan. 25, 2010

(30) Foreign Application Priority Data
Jul. 24, 2007 (IE) S2007/0534

(51) Int. Cl. A61B 17/56

(52) U.S. Cl. 606/103

(57) ABSTRACT

The present invention provides a jig for use in relation to the surgical treatment of a bone having a bone fracture, the jig being formed to receive at least part of the bone about the bone fracture; said jig (10) having first (24,24′) and second (32) sets of apertures; said first set of apertures being located distal to the bone fracture; and said second set of apertures being located proximal to the bone fracture. The jig facilitates the positioning of intramedullary wires in the fixation of fractures to the bone, such as, but not limited to, fractures of the olecranon, for example, a Type II fracture of the olecranon.

The present invention relates to a jig. In particular, the invention finds utility in the field of orthopaedic surgery as a jig for use in relation to fractures of the bone, such as fractures of the olecranon. However, it is not intended that the invention be limited solely to fractures of the olecranon.

The olecranon is the proximal bony projection of the ulna at the elbow. Olecranon fractures are a diverse group of injuries, ranging from simple non-displaced fractures to complex fracture dislocations of the elbow joint. The most common causes of an olecranon fracture are a fall on the semi-flexed supinated forearm, or direct trauma, as in falls on, or blows to, the point of the elbow.

Fractures of the olecranon are generally divided into three types, depending on the stability of the joint and the amount of separation among the broken pieces of bone. The treatment options are dependent on the type of fracture (Mayo Classification):
Type I fractures are generally stable with little displacement, and can generally be treated non-surgically, usually with a splint or sling to hold the elbow at a 90° angle.
Type II fractures are the most common. They are relatively stable, although there is displacement of the bone pieces, and are best treated surgically. A plate or combination of wires and pins/screws is used to hold the bones in place.
Type III fractures are displaced and involve more than 50% of the joint surface, resulting in joint instability. These fractures are treated surgically, usually with a plate that fits under the ulna and around the tip of the elbow, and is held in place by screws.

Tension Band Wiring (TBW) is the most common fixation technique for simple (Type II) fractures. The goal is to convert the extensor force of the triceps to a dynamic compression force along the articular surface in order to maximise functional outcome. In this technique, a direct straight posterior incision is used with the patient supine with the arm across the chest or, occasionally, in the prone position. Usually two smooth Kirschner (K-) wires that are approximately 1.0-2.0 mm in diameter are placed through the triceps tendon into the olecranon, down the longitudinal axis of the ulna, or through the anterior cortex of the ulna. A transverse drill hole through the ulna at 1-2 cm distal to the articular surface provides the distal fixation point. A figure-of-eight cerclage wire is passed through the transverse drill-hole in the ulna, and is subsequently inserted into the triceps tendon, tensioned and secured.

This technique transmits tension provided from muscular flexion through the cerclage wire, causing the intra-articular component of the fracture to undergo compression, thereby facilitating bone repair.

However, poorly located intramedullary wires are prone to “backing-out”. Resulting protrusion can hinder forearm rotation and may cause damage to nerves or arteries. Therefore, accurate positioning of intramedullary wires is essential for the TBW technique to function correctly, to facilitate bone repair, and to speed the recovery process.

It is an object of the present invention to provide a jig for the positioning of intramedullary wires in the fixation of fractures to the bone, such as, but not limited to, fractures of the olecranon, for example, a Type II fracture of the olecranon.

According to a first aspect of the present invention there is provided a jig for use in relation to the surgical treatment of a bone having a bone fracture, the jig being formed to receive at least part of the bone about the bone fracture; said jig having first and second sets of apertures; said first set of apertures being located distal to the bone fracture; and said second set of apertures being located proximal to the bone fracture.

Optionally, the apertures of the first set of apertures are aligned with each other so that, in use, a drill bit can be passed through the first set of apertures. Further optionally, the apertures of the second set of apertures are aligned substantially parallel with each other. Still further optionally, the apertures of the second set of apertures are arranged so that, in use, any fracture-fixing implements, for example wires, passing therethrough will enter the intramedullary canal of the fractured bone.

Optionally, the jig is shaped and dimensioned to receive at least part of an appendage, for example an arm, of a patient, the at least part of the appendage, for example an arm, having a fractured bone.

Preferably, the jig is arranged such that, in use, the terminal ends of the jig are located on opposing sides of the bone fracture. In the case of a fracture of the olecranon, the proximal terminal end of the jig is located adjacent the olecranon, and the distal terminal end is located adjacent the shaft of the ulna bone.

Preferably, the first set of apertures is oriented to be substantially perpendicular to the second set of apertures.

Optionally, the first set of apertures comprises one or more pairs of apertures, wherein the or each pair defines a rectilinear axis between the pair of apertures. In the case of a fracture of the olecranon, preferably the rectilinear axis defined between each pair of first set of apertures extends substantially perpendicular to the respective longitudinal axis of the ulna bone. Preferably, the or each pair of first set of apertures are arranged in series along the longitudinal length of the jig.

Preferably, the second set of apertures comprises a plurality of apertures. Preferably, the second set of apertures comprises at least two apertures; optionally three or at least three apertures. In the case of a fracture of the olecranon, preferably the longitudinal axis of each of the apertures of the second set of apertures extends substantially parallel to the longitudinal axis of the ulna bone.

Preferably, the jig comprises a first component and a second component. Preferably, the first component is a reference component and the second component is an aiming component, for aiming the fracture-fixing implements therethrough.

Preferably, the first component includes the first set of apertures.

Preferably, the second component includes the second set of apertures.

Preferably, some apertures can be used as a drill guide. Optionally, some apertures can be used as a reference guide. Further optionally, some apertures can be used as a guide for surgical instrumentation.

Preferably, the first and second components are adjustably arranged relative to each other.

Preferably, the first and second components are slidably arranged relative to each other, to facilitate relative motion. Further preferably, the first and second components are slidably mountable to one another and arranged for relative reciprocal movement. It is also understood that any mechanism can be utilised by which relative movement of the first and second components is facilitated.

Preferably, the second component comprises means to reversibly mount the first component thereto, and demount the first component therefrom. Preferably, the means comprise a guide oriented substantially parallel to the longitudinal axis of the second component. Optionally, the guide comprises a pair of spaced apart elongate arms. Preferably, the mounting means comprises slide projections dimensioned and arranged for relative sliding movement with correspondingly dimensioned guide channels of the first component. Preferably, the first component comprises means to reversibly engage the second component. Preferably, the engaging means is shaped and dimensioned to reversibly engage with the mounting means of the second component. More preferably, the engaging means comprises guide channels dimensioned and arranged for relative sliding movement with the slide projections provided on the second component.

Preferably, the second component is shaped and dimensioned to traverse the bone fracture site. Further preferably, the second component is generally “L-shaped” in side view, wherein the terminal ends of the elongate arms of the second component are located on opposing sides of the bone fracture, and extend proximally and distally about the bone fracture site. In the case of a fracture of the olecranon, preferably the proximal terminal end of the elongate arms of the second component is located adjacent the humerus bone, and the terminal distal end of the elongate arms provide the means to reversibly mount the first component thereto. Accordingly, the first component is, in use, reversibly mounted to the distal ends of the arms of the second component.

Preferably, the second set of apertures is located adjacent the terminal proximal end of the second component.

Preferably, each of the first and second sets of apertures is shaped and dimensioned to reversibly receive a drill bit.

Preferably, a spike projection is provided adjacent the terminal proximal end of the second component. Preferably, the spike projection extends substantially perpendicular from the appendage-engaging face of the second component. Preferably, the spike projection engages with at least part of the appendage to facilitate increased stability.

Preferably, in use, the first component is shaped and dimensioned to at least partially surround at least part of a fractured bone. Optionally, in use, the first component is shaped and dimensioned to partially surround at least part of an appendage, for example an arm, of a patient, the at least part of the appendage, for example an arm, having a fractured bone.

Preferably, the first set of apertures comprises at least one pair of apertures wherein the or each pair defines a rectilinear axis between them, and the first component is shaped and dimensioned such that, in use, the or each rectilinear axis extends through, but substantially perpendicular to the longitudinal axis of, the fractured bone. In the case of a fracture of the olecranon, preferably the rectilinear axis defined between the or each pair of first set of apertures extends substantially perpendicular to the longitudinal axis of the ulna bone.

Preferably, each pair of first sets of apertures is shaped and dimensioned to reversibly receive a drill bit.

Preferably, the first component further comprises a guide aperture. Preferably, the guide aperture is shaped and dimensioned to allow a surgical implement to pass therethrough. Further preferably, the guide aperture is oriented substantially perpendicular to the longitudinal axis of the fractured bone.

Optionally, the first component further comprises means for temporarily inhibiting the relative motion of the first component relative to the second component. Optionally, the inhibiting means are adapted to allow for adjustable movement of the first component relative to the second component. Preferably, the inhibiting means comprises one or more openings, wherein each opening is shaped and dimensioned to reversibly receive a plug. Optionally, the plug is a rigid body, such as a surgical instrument. Optionally, the second component is adapted to inhibit the relative movement of the first component. Preferably, the second component further comprises a stop. Preferably, the stop is arranged to reversibly engage with the plug. Further preferably, the stop is a cross-member, which inhibits the reciprocal movement of the first component relative to the second component by inhibiting the passage of the plug thereby.

Preferably, the openings are arranged in a series in the direction of reciprocal motion of the first component relative to the second component.

Preferably, the jig is formed from a sterilisable material.

The jig can be formed from a rigid, lightweight material such as plastic.

Alternatively, it can be formed of surgical stainless steel.

According to a second aspect of the present invention, there is provided a method for the fixation of a bone having a bone fracture, the method comprising the steps of positioning a jig according to the first aspect of the invention on the bone, whereby the first set of apertures is located distal to the bone fracture and the second set of apertures is located proximal to the bone fracture; drilling at least one hole distal to the bone fracture through the first set of apertures; reducing the bone fracture; and inserting fracture-fixing implements through the second set of apertures. Optionally, the fracture-fixing implements are intramedullary wires.

Optionally, the method also comprises the step of drilling holes proximal to the bone fracture through the second set of apertures, after the fracture-reducing step.

Preferably, the method comprises the steps of positioning the first component on the bone distal the bone fracture; positioning a drill bit between the first set of apertures; reducing the bone fracture; positioning the second component relative to the first component; and inserting fracture-fixing implements through the second set of apertures. Optionally, the fracture-fixing implements are intramedullary wires.

Optionally, the method further comprises the step of temporarily inhibiting the movement of the second component relative to the first component, after the second component-positioning step.

Optionally, the method also comprises the additional step of drilling holes proximal to the bone fracture site through the second set of apertures, after the second component-positioning step.

Preferably, the intramedullary wires are selected from metal K-wires, or from bioabsorbable pins such as Trim-It™ pins. The use of intramedullary wires such as K-wires, or bioabsorbable pins such as Trim-It™ pins can negate the optional requirement for predrilled holes proximal to the bone fracture site, as this form of intramedullary wire can be both self-drilling and self-securing.

Preferably, the method comprises the further steps of removing the second component; removing the drill bit; removing the first component; and, optionally, further fixing the fracture.

The further fixation step can comprise a figure-of-eight arrangement. The figure-of-eight arrangement can be formed from metal cerclage wire or from suture material such as FiberTape™.

An embodiment of the invention will now be described, by way of example, with reference to the accompanying drawings, in which:

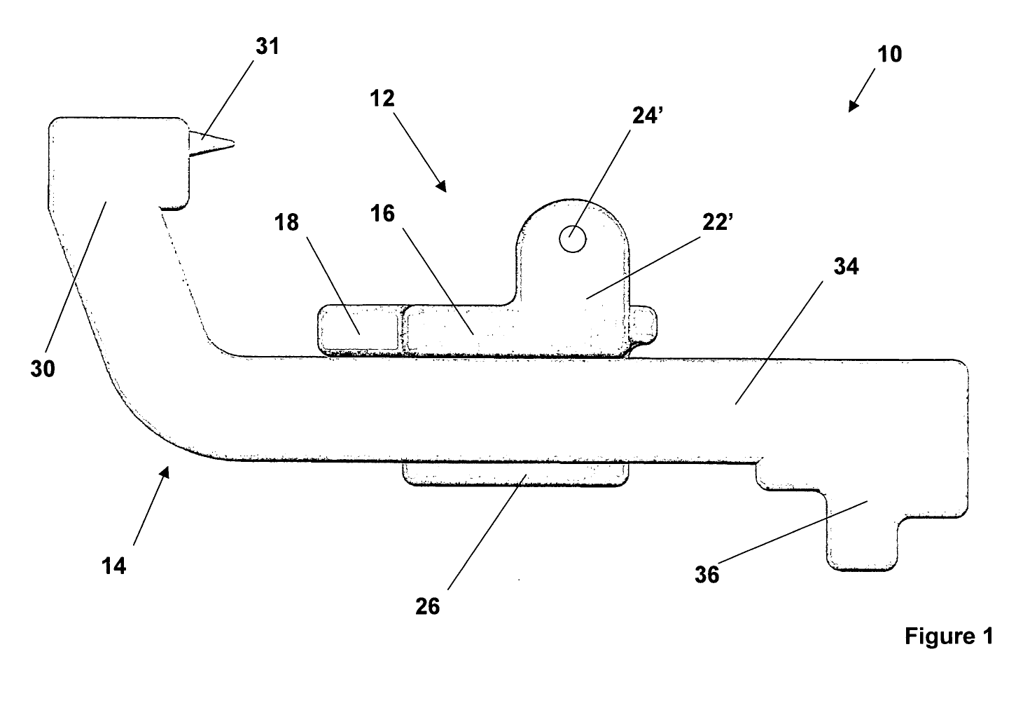

FIG. 1 is a side view of a jig according to a preferred embodiment of the present invention;

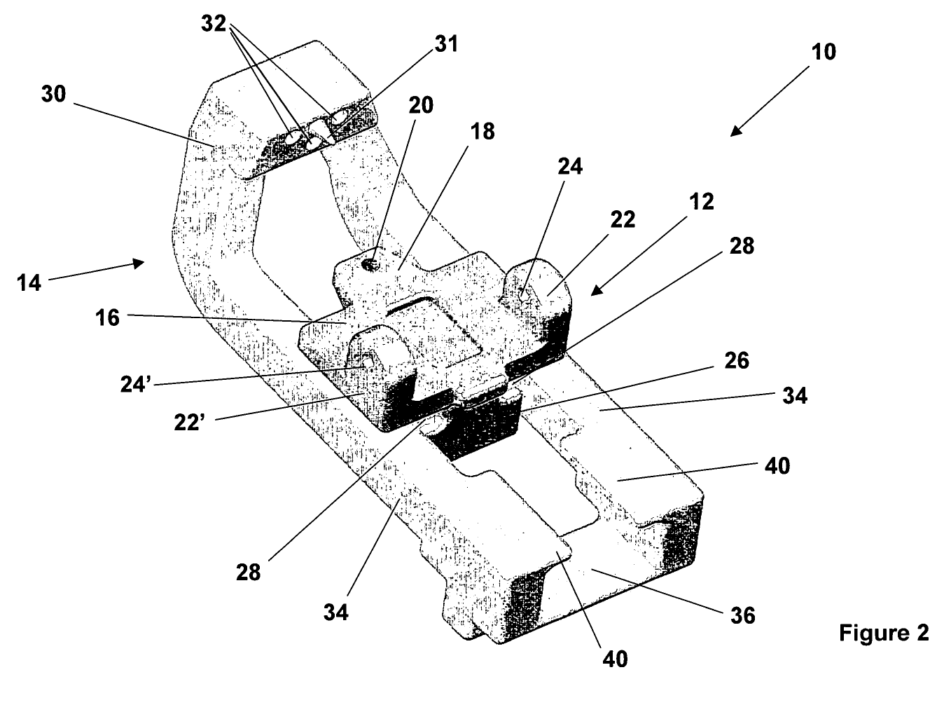

FIG. 2 is a perspective view of the jig of FIG. 1;

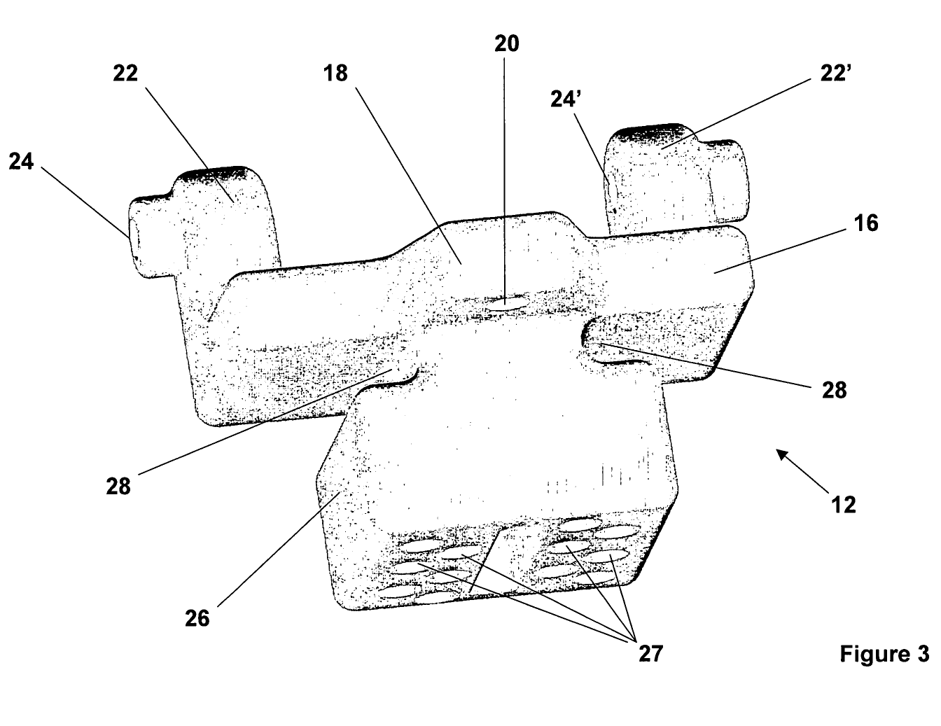

FIG. 3 is a perspective view of a first component of the jig of FIG. 2;

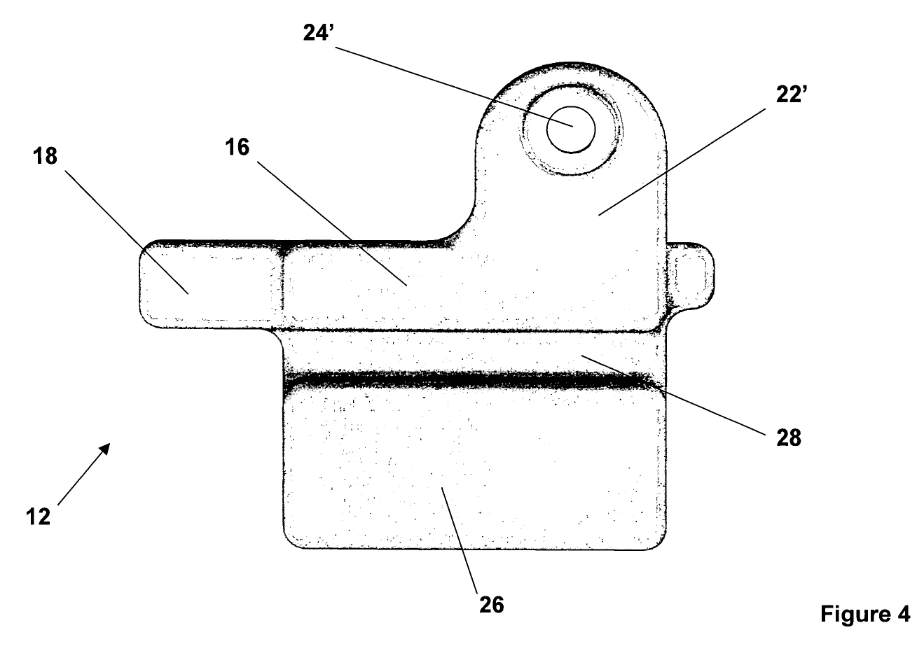

FIG. 4 is a side view of the first component of FIG. 3;

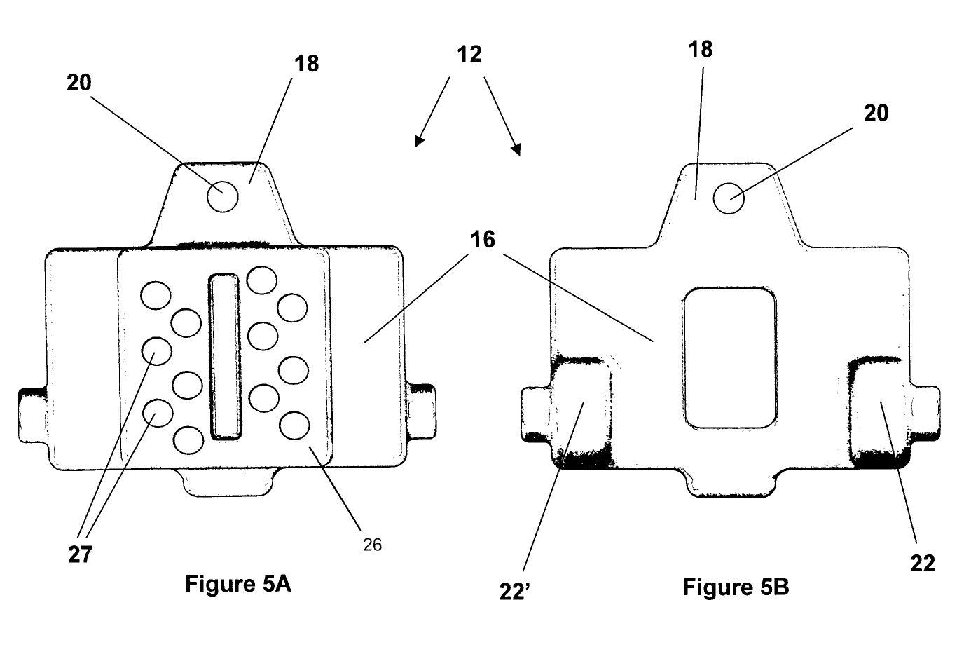

FIG. 5A is a plan view of the first component of FIG. 3;

FIG. 5B is a plan view of the underside of the first component of FIG. 5A;

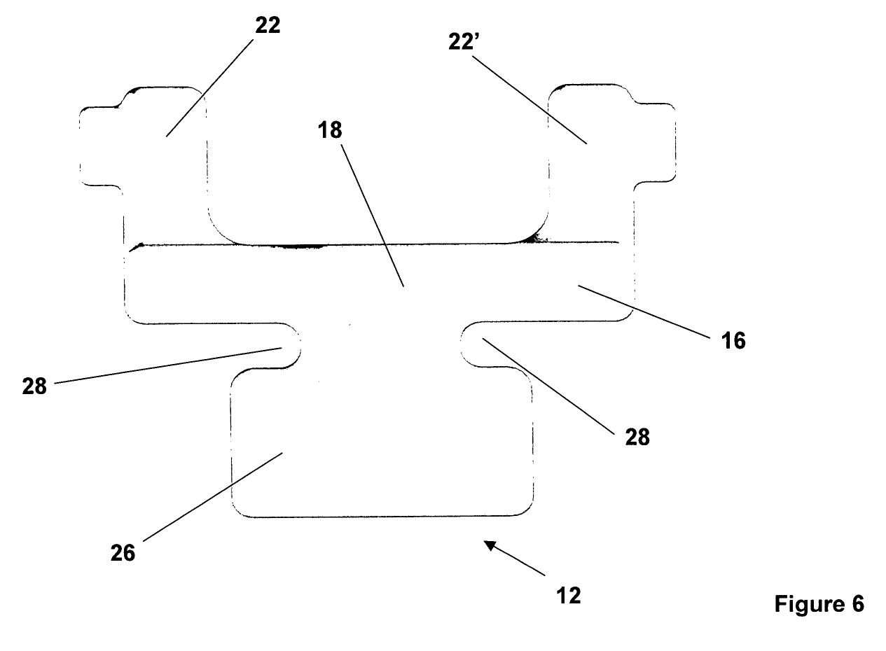

FIG. 6 is an end view of the first component of FIG. 3;

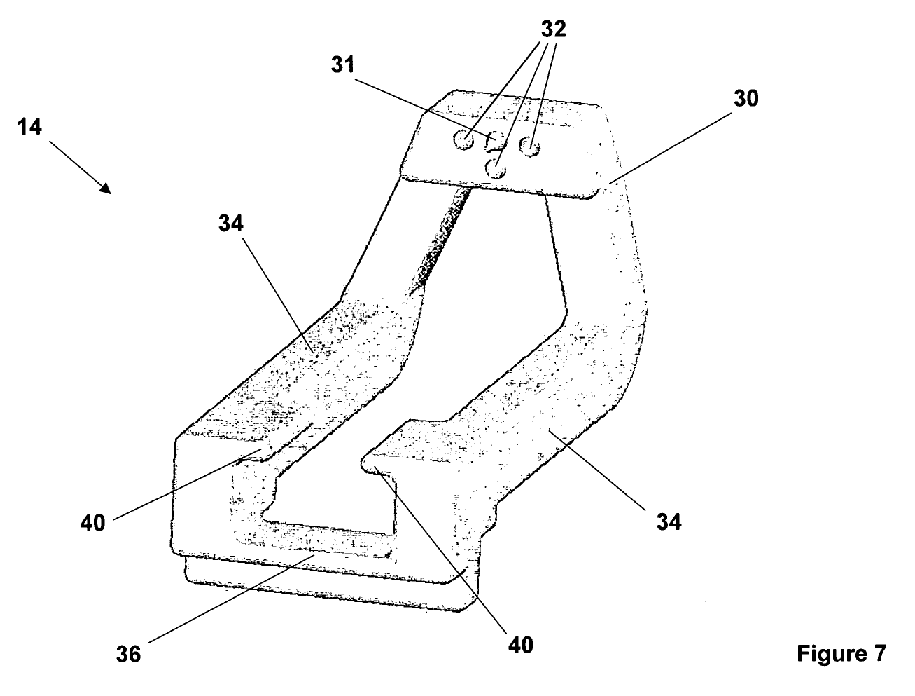

FIG. 7 is a perspective view of a second component of the jig of FIG. 2;

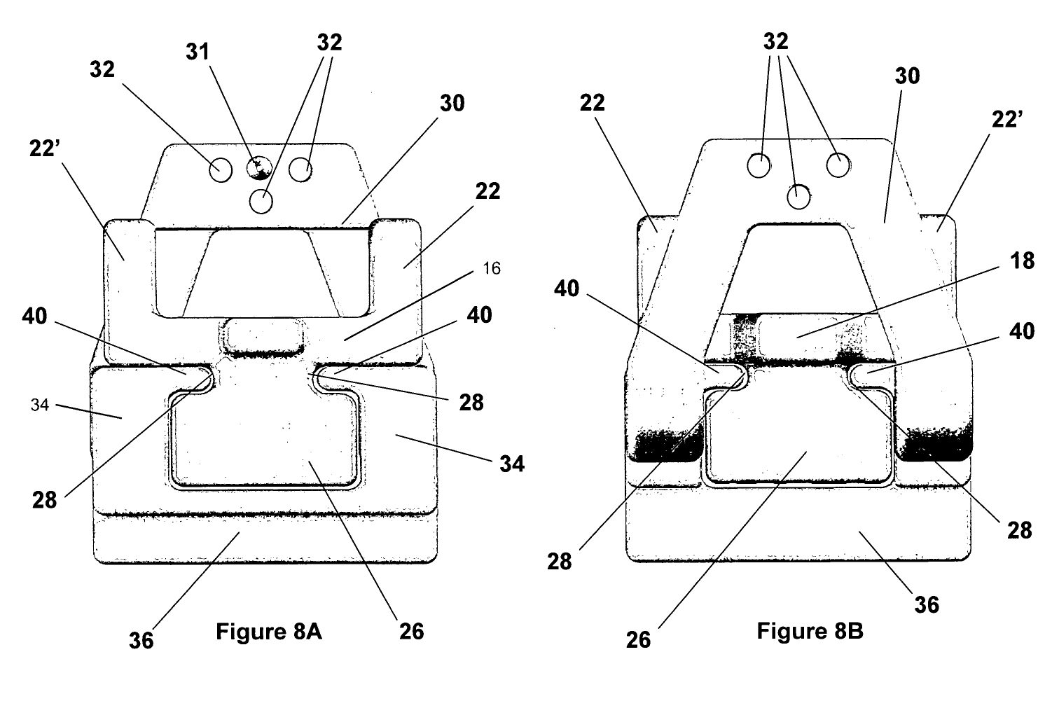

FIG. 8A is a first end view of the jig of FIG. 2;

FIG. 8B is a second end view of the jig of FIG. 8A;

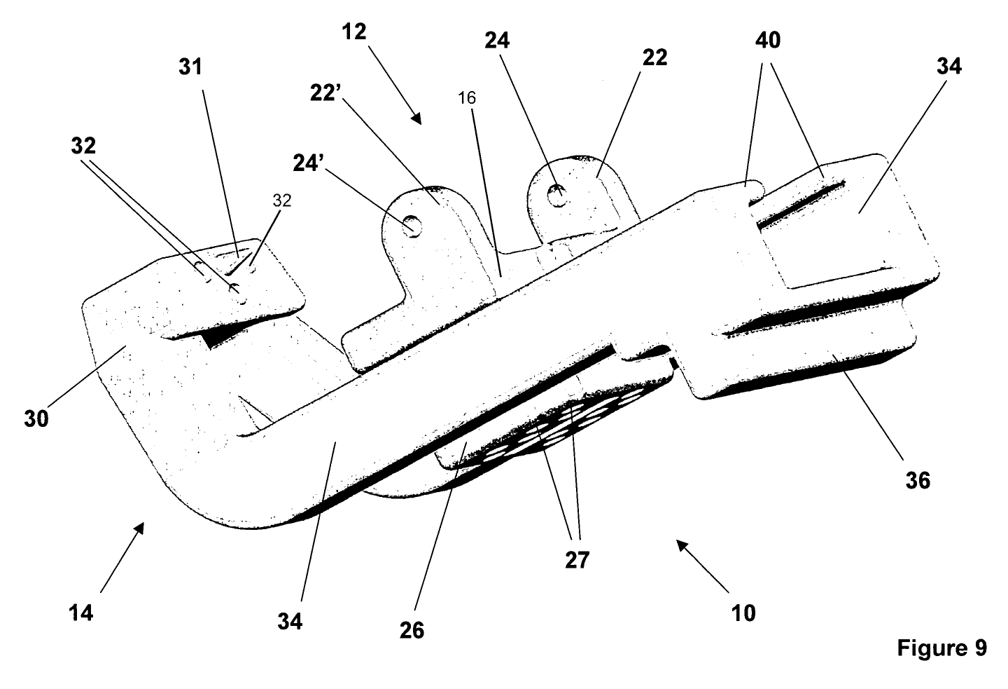

FIG. 9 is a perspective view of the underside of the jig of FIG. 1; and

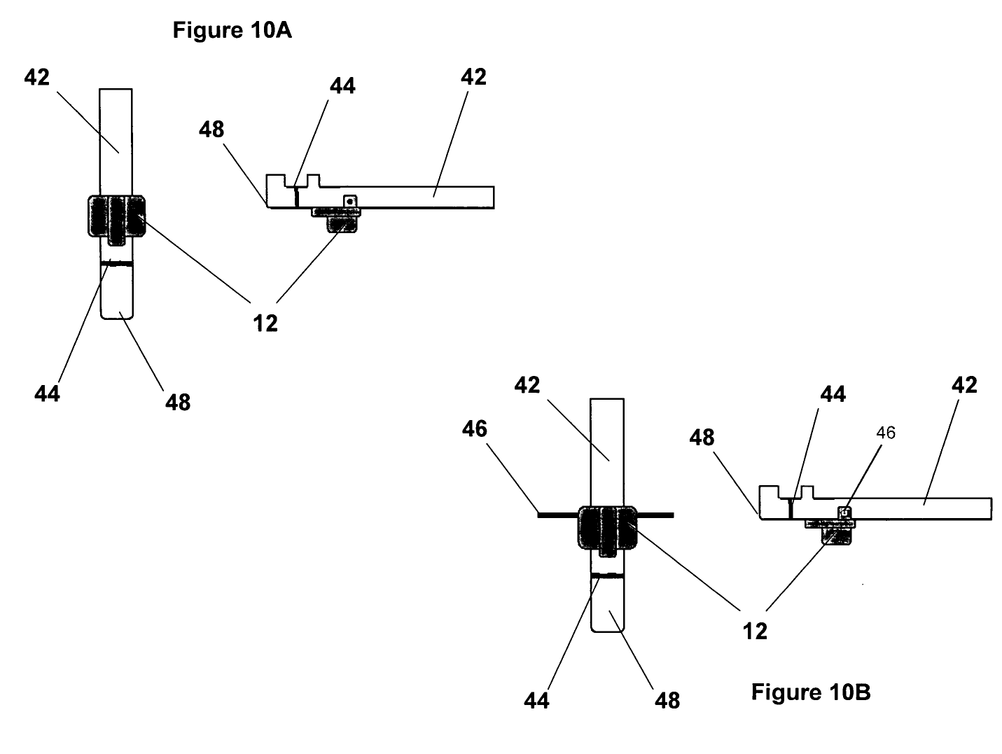

FIGS. 10-12 are schematic diagrams depicting a method of use of the jig of FIG. 1.

Referring now to FIGS. 1 and 2 of the drawings, there is shown a jig 10 according to a preferred embodiment of the present invention. The jig 10 comprises a first component 12, and a second component 14.

With reference to FIGS. 3-6, there is shown a first component 12 of the jig 10. The first component 12 comprises a substantially planar stage 16, which is generally parallelepiped in shape. A proximal reference projection 18 extends from the proximal end, and is continuous with, the planar stage 16. A guide aperture 20 is provided which extends perpendicularly through the proximal reference projection 18. The guide aperture 20 facilitates the accurate positioning of the first component 12 by allowing visualisation of the central longitudinal axis of the first component 12 relative to the fractured bone, or appendage having the fractured bone (not shown). The guide aperture 20 is also shaped and dimensioned to allow the passage of, for example, a forceps therethrough, to facilitate reduction of the fracture. Two side projections, one medial 22, and one lateral 22′, are provided, each of which extend from respective sides of the planar stage 16. Each side projection 22, 22′ is substantially perpendicular to the plane of the planar stage 16. A drill-guide aperture 24, 24′ is provided on each side projection 22, 22′, and extends perpendicularly through the respective side projection 22, 22′. The drill-guide apertures 24, 24′ comprise the first set of apertures. An engaging projection 26 extends substantially perpendicular from, in use, a lower face of the planar stage 16, in the opposite direction to each of the side projections 22, 22′. The engaging projection 26 is generally parallelepiped in shape. A set of guide channels 28 is provided along the longitudinal axis of the neck of the engaging projection 26. A series of paired openings 27 are provided in the engaging projection 26, see also FIG. 9, and each opening 27 is arranged to receive a plug, such as a rigid body, for example a surgical instrument (not shown).

FIG. 7 is a perspective view of a second component 14 of the jig 10. The second component 14 comprises a proximal block 30, which is generally “U-shaped” with respective ends. Guide apertures 32 (or second set of apertures) are provided towards the bridge of the proximal block 30, each of which guide apertures 32 runs substantially parallel to the longitudinal axis of the second component 14. A spike projection 31 is provided at the bridge of the proximal block 30, and is oriented to, in use, engage with the humerus bone, or appendage having the humerus bone (not shown), to increase stability of the second component 14 relative to the humerus bone (not shown). A pair of elongate arms 34 extends distally from the respective ends of the proximal block 30 and is generally perpendicular to the plane of the proximal block 30. The pair of elongate arms 34 together define a guide. The space between the elongate arms 34 is shaped and dimensioned such that the engaging projection 26 of the first component 12 can pass therebetween, FIG. 9. A cross-member 36 is provided at the distal end of each elongate arm 34 to provide structural support. A slide projection 40, is provided on the internal face of each of the elongate arms 34. Each slide projection 40 extends substantially perpendicularly from the longitudinal axis of each arm 34, and is shaped and dimensioned to reversibly engage with the set of guide channels 28 provided along the longitudinal axis of the neck of the engaging projection 26 of the first component 12.

As depicted in FIGS. 8A, 8B, and 9, once assembled, the engaging projection 26 of the first component 12 locates between each of the elongate arms 34 of the second component 14. Each of the slide projections 40 of the second component 14 locates within each of the complementary guide channels 28 of the first component 12, thereby allowing the first component 12 to move reciprocally along a defined length of the second component 14. When the slide projections 40 of the second component 14 and the guide channels 28 of the first component 12 are engaged, the engaging projection 26 of the first component 12 is inhibited from disengaging from the second component 14.

FIGS. 10-12 are schematic diagrams depicting a method of use of the jig 10, wherein the left panel of each figure depicts a plan view, and the right panel of each figure depicts a side view.

Referring firstly to FIG. 10A, following surgical exposure, the first component 12 is placed on the posterior border of the ulna 42, distal to the bone fracture 44. The proximal reference projection 18 and engaging projection 26, FIGS. 3-6, are positioned over the longitudinal axis of the ulna 42 using the guide aperture 20. Each of the side projections 22, 22′, FIGS. 3-6, is located on either side of the ulna shaft 42. Referring now to FIG. 10B, once the surgeon has positioned the first component 12, a 2 mm transverse drill hole is drilled using a 2 mm drill-bit 46, guided by the drill-guide apertures 24, 24′ of each of the side projections 22, 22′, FIGS. 3-6. The drill-bit 46 passes through the first drill-guide aperture 24, through both cortices of the ulna and exits through the complementary drill-guide aperture 24′ (or vice versa). The drill bit 46 can then be uncoupled from the drill and temporarily left in-situ to secure the first component 12 in position. The fracture can be reduced with a bone reduction forceps (not shown), distally using the guide aperture 20, FIGS. 3-6, and proximally in the olecranon fragment 48.

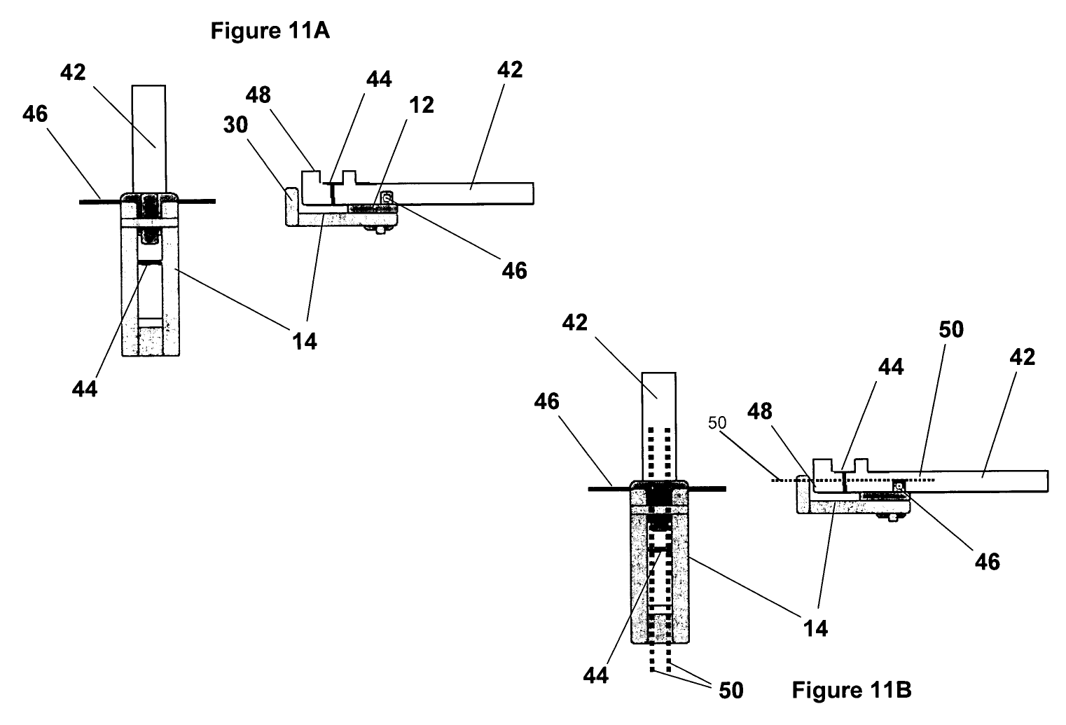

Referring to FIG. 11A, once the fracture 44 is reduced satisfactorily, the second component 14 is mounted to the first component 12, by passing the guide channels 28 of the engaging projection 26 through the slide projections 40 of the second component 14, FIG. 9. The second component 14 is then slid reciprocally along the first component 12, such that the proximal block 30 is located adjacent the olecranon fragment 48. The spike projection 31 engages with the olecranon fragment 48, humerus bone, or appendage (not shown) to increase the stability of the proximal block 30 relative to the humerus bone or appendage. A plug, such as a forceps (not shown), can be inserted into an appropriate opening 27 of the first component 12, FIG. 9, such that the plug extends beyond the face of the engaging projection 26 of the first component 12. The plug (not shown) engages with the cross-member 36 of the second component 14 to inhibit the first component 12 from moving relative to the second component 14.

Referring to FIG. 11B, at least two 2mm intramedullary wires 50 can then be inserted through the guide apertures 32, FIG. 7, of the proximal block 30 of the second component 14. The intramedullary wires are then directed by the guide apertures 32 along the intramedullary canal of the ulna 42 at the specific correct depth and direction.

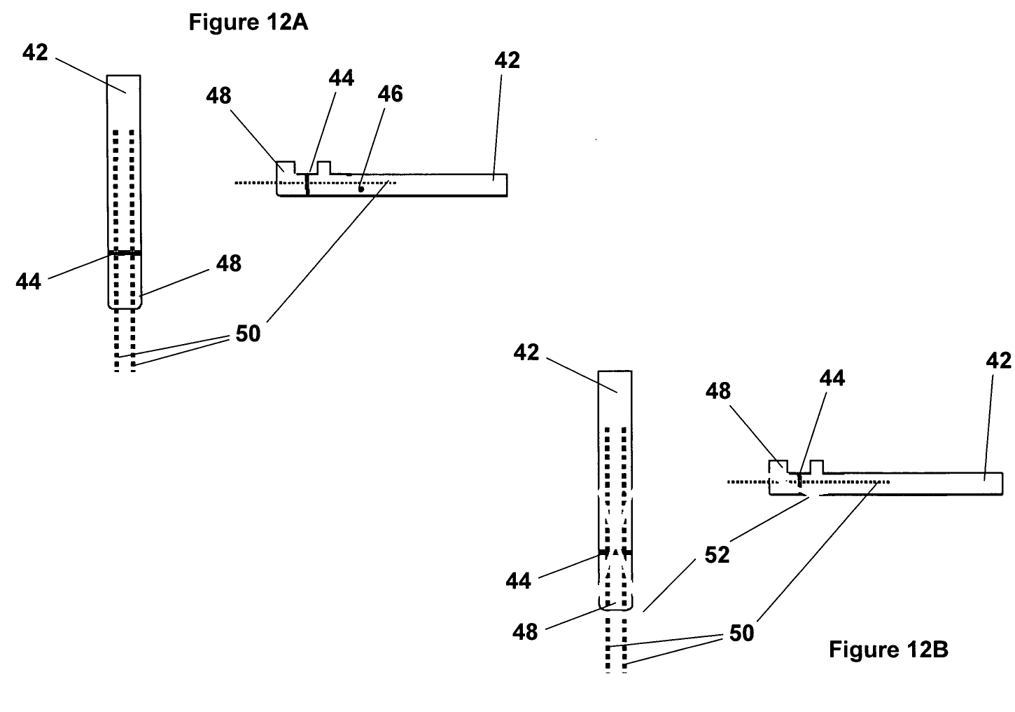

Once the intramedullary wires 50 have been inserted, the bone reduction forceps can be removed (temporarily, if need be), and the plug (not shown) can be removed from the opening 27 of the first component 12, to facilitate removal of the second component 14, by sliding the second component 14 away from the first component 12 such that each slide projection 40 of the second component 14 disengages from the set of guide channels 28 of the first component 12, thereby allowing the engaging projection 26 of the first component 12 to pass between the elongate arms 34 of the second component 14. The intramedullary wires 50 can be further driven home, if required. The 2mm transverse drill-bit 46 is removed, thereby facilitating removal of the first component 12, FIG. 12A.

Referring now to FIG. 12 B, there is shown a schematic diagram depicting a further method of use of the jig 10. A cerclage wire/suture 52 is threaded through the transverse drill-hole and deep to the triceps tendon (not shown) in a figure-of-eight fashion and is tensioned and tied. The intramedullary wires 50 are then cut flush with the olecranon 48.

1-34. (canceled)

35. A jig for use in relation to the surgical treatment of a bone having a bone fracture, the jig being formed to receive at least part of the bone about the bone fracture; said jig having first and second sets of apertures; said first set of apertures being located distal to the bone fracture; and said second set of apertures being located proximal to the bone fracture.

36. A jig according to claim 35, wherein the apertures of the second set of apertures are aligned substantially parallel with each other.

37. A jig according to claim 35, wherein the apertures of the second set of apertures are arranged so that, in use, any fracture-fixing implements, for example wires, passing therethrough will enter the intramedullary canal of the fractured bone.

38. A jig according to claim 35, wherein the jig is arranged such that, in use, the terminal ends of the jig are located on opposing sides of the bone fracture.

39. A jig according to claim 35, wherein the first set of apertures is oriented to be substantially perpendicular to the second set of apertures.

40. A jig according to claim 35, wherein the first set of apertures comprises at least one pair of apertures, wherein the at least one pair defines a rectilinear axis between the pair of apertures; and the at least onepair of first set of apertures are arranged in series along the longitudinal length of the jig.

41. A jig according to claim 35, wherein the second set of apertures comprises at least two apertures.

42. A jig according to claim 35, wherein the jig comprises a first component and a second component.

43. A jig according to claim 42, wherein the first component includes the first set of apertures, and the second component includes the second set of apertures.

44. A jig according to claim 42, wherein the first and second components are adjustably arranged relative to each other.

45. A jig according to claim 42, wherein the first and second components are slidably mountable to one another and arranged for relative reciprocal movement.

46. A jig according to claim 42, wherein the second component comprises means to reversibly mount the first component thereto, and demount the first component therefrom.

47. A jig according to claim 46, wherein the mounting means comprises slide projections dimensioned and arranged for relative sliding movement with the first component.

48. A jig according to claim 42, wherein the first component comprises means to reversibly engage the second component.

49. A jig according to claim 48, wherein the engaging means comprises guide channels dimensioned and arranged for relative sliding movement with the slide projections provided on the second component.

50. A jig according to claim 42, wherein the second component is shaped and dimensioned to be located on opposing sides of the bone fracture, and extend proximally and distally about the bone fracture site.

51. A jig according to claim 42, wherein the second set of apertures is located adjacent the terminal proximal end of the second component.

52. A jig according to claim 42, further comprising a spike projection adjacent the terminal proximal end of the second component.

53. A jig according to claim 42, wherein, in use, the first component is shaped and dimensioned to at least partially surround at least part of a fractured bone.

54. A jig according to claim 42, wherein the first component further comprises a guide aperture, the guide aperture being shaped and dimensioned to allow a surgical implement to pass therethrough, and oriented substantially perpendicular to the longitudinal axis of the fractured bone.

55. A jig according to claim 42, wherein the first component further comprises means for temporarily inhibiting the relative motion of the first component relative to the second component.

56. A jig according to claim 55, wherein the inhibiting means comprises at least one opening, each opening being shaped and dimensioned to reversibly receive a plug.

57. A jig according to claim 56, wherein the openings are arranged in a series in the direction of reciprocal motion of the first component relative to the second component.

58. A jig according to claim 42, wherein the second component is adapted to inhibit the relative movement of the first component.

59. A jig according to claim 56, wherein the second component further comprises a stop, the stop being arranged to reversibly engage with the plug.

60. A method for the fixation of a bone having a bone fracture, the method comprising the steps of positioning a jig according to claim 35 on the bone, whereby the first set of apertures is located distal to the bone fracture and the second set of apertures is located proximal to the bone fracture; drilling at least one hole distal to the bone fracture through the first set of apertures; reducing the bone fracture; and inserting fracture-fixing implements through the second set of apertures.

61. A method according to claim 60, wherein the method also comprises the step of drilling holes proximal to the bone fracture through the second set of apertures, after the fracture-reducing step.

62. A method according to claim 60, wherein the method comprises the steps of positioning a jig according to claim 8 on the bone, wherein the first component is positioned on the bone distal the bone fracture; positioning a drill bit between the first set of apertures; reducing the bone fracture; positioning the second component relative to the first component; and inserting fracture-fixing implements through the second set of apertures.

63. A method according to claim 62, wherein the method further comprises the step of temporarily inhibiting the movement of the second component relative to the first component, after the positioning step.

64. A method according to claim 62, wherein the method also comprises the additional step of drilling holes proximal to the bone fracture site through the second set of apertures, after the second component-positioning step.

65. A method according to claim 60, wherein the fracture-fixing implements are intramedullary wires.

66. A method according to claim 65, wherein the intramedullary wires are selected from metal K-wires.

67. A method according to claim 62, wherein the method comprises the further steps of removing the second component; removing the drill bit; and removing the first component.

68. A method according to claim 67 further comprising the step of further fixing the fracture, wherein the further fixation step comprises a figure-of-eight arrangement formed from metal cerclage wire.
